(19) United States
(12) Patent Application Publication
(10) Pub. No.: US 2024/0261530 A1
(43) Pub. Date: Aug. 8, 2024

(54) MEDICAL APPARATUS FOR SUPPLYING GAS, SUCH AS NO, EQUIPPED WITH A DEVICE FOR FIXING TO AN ELONGATE SUPPORT

(71) Applicant: INOSYSTEMS, Antony (FR)

(72) Inventors: Nicolas TIRILLY, Antony (FR); Christian KAHALE, Antony (FR)

(21) Appl. No.: 18/420,922

(22) Filed: Jan. 24, 2024

(30) Foreign Application Priority Data
Feb. 3, 2023 (FR) 2301039

(51) Int. Cl. A61M 16/14

(52) U.S. Cl. CPC A61M 16/14; A61M 2202/0275; A61M 2209/082

(57) ABSTRACT

The invention relates to a medical apparatus (1) for supplying gas, such as gaseous NO, comprising a shell (2) and a device (10), arranged on said shell (2), for fixing the latter to an elongate support (S). The fixing device (10) comprises a clamp body (12) and a clamp element (13) movable by pivoting (XX) relative to each other, which are configured as a first jaw (15) and a second jaw (16) forming a clamp. A rotary manipulation member (11), which can be actuated in rotation, allows the jaws (15, 16) to be moved towards or away from each other. It comprises a head (11.1) intended to be grasped manually by the user, and an actuation rod (11.2) with a threaded portion (11.4) cooperating with an internally threaded actuation part in order to move the jaws (15, 16) towards or away from each other. A torque-limiting system (20) is arranged in the head (11.1) of the rotary manipulation member (11). It comprises a stack of spring washers (21) cooperating with a pressure washer (24).

### CROSS-REFERENCE TO RELATED APPLICATION

This patent application claims priority to FR 2301039, filed Feb. 3, 2023 and the entire contents of which are incorporated herein by reference.

### FIELD OF ART

The invention relates to a medical apparatus for supplying gas, such as NO, typically an NO/N2 gas mixture, equipped with a fixing device making it possible to fix, that is to say attach and hold, the apparatus to an elongate support, in particular of the rod or bar type.

### BACKGROUND

During their use, some medical apparatuses have to be fixed, that is to say attached and held in position, on supports having different structures, typically supports of the rod or bar type having square, rectangular or cylindrical cross sections.

Thus, some medical apparatuses for supplying gases, such as those for supplying NO, i.e. typically an NO/N2 mixture, must be able to be transported in emergency vehicles, such as an ambulance, an airplane or the like. During transport, in order to prevent them from falling, they must be firmly fixed to a support arranged in the vehicle (e.g. a rectangular or square rod) or on the stretcher (e.g. a cylindrical bar) carrying the patient to whom gas, e.g. NO, is administered by means of these apparatuses. An example of an NO delivery apparatus is described in EP-A-3906955.

To this end, a dedicated fixing device, for example a clamp device, can be fitted on the rear wall of the external casing of the medical apparatus in question, that is to say on its peripheral shell. Thus, EP-A-3639975 teaches a clamping device making it possible to fix an apparatus to supports of different sizes and shapes, in particular a medical apparatus in a hospital environment. The device is formed of a bottom part comprising a concave lower jaw and a rear extension forming a first clamping handle, and of an upper part comprising a concave upper jaw and a rear portion serving as a second handle. These parts carry a plurality of pivot pins enabling the two parts to pivot. Two pins also serve as anchor points for a locking arm serving to adjust the spacing of the jaws. A rotary adjustment wheel arranged around the locking arm makes it possible to adjust the length thereof, hence the spacing of the jaws. However, such a clamp structure is complex and not very easy to manipulate; in particular, the presence of the rotary adjustment wheel complicates its architecture.

A problem is therefore to propose a medical apparatus for supplying gas, in particular NO, equipped with a fixing device making it possible to fix the medical apparatus to an elongate support of the rod or bar type, avoiding or limiting all or some of the problems, risks and/or disadvantages mentioned above, regardless of whether the support is horizontal, vertical or oblique.

### SUMMARY

A solution of the invention relates to a medical apparatus for supplying gas, in particular gaseous NO, such as an apparatus for supplying an NO/N2 gas mixture, comprising a shell having an outer wall and a fixing device, arranged on the outer wall of said shell, for fixing the latter to an elongate support, wherein the fixing device comprises:
a clamp body and a clamp element movable by pivoting relative to each other,
at least part of the clamp body being configured as a first jaw and at least part of the movable clamp element being configured as a second jaw, said first and second jaws being arranged face to face in order to form a clamp, and
a rotary manipulation member, which can be actuated in rotation by a user, cooperating with the clamp element, when actuated, in order to move the clamp element with respect to the clamp body and move the first jaw towards or away from the second jaw.

In addition:
the rotary manipulation member comprises a head intended to be grasped manually by the user, and an actuation rod comprises a threaded portion, and
an internally threaded actuation part cooperates with the threaded portion of the actuation rod and with the clamp element in order to effect the pivoting of the clamp element relative to the clamp body in such a way as to move said first and second jaws towards or away from each other.

According to the invention, the rotary member further comprises a torque-limiting system arranged in the head of the rotary manipulation member, said torque-limiting system comprising a stack of spring washers cooperating with a pressure washer.

Depending on the embodiment considered, the medical apparatus of the invention can comprise one or more of the following features:
the clamp body is fixed.
the clamp body is fixed to a support plate.
the clamp body and the clamp element are carried by a support plate.
the support plate is fixed to the outer wall of said shell, preferably fixed by screwing.
the support plate comprises a perforated plate.
the support plate is fixed to the rear face of the shell of the apparatus.
the threaded portion of the actuation rod is arranged at a free end of the actuation rod of the rotary manipulation member.
the threaded portion of the actuation rod comprises a peripheral thread, that is to say a thread carried by a part of the peripheral surface of the actuation rod.
the actuation rod has a cylindrical shape.
the actuation rod is made of metal, such as stainless steel.
the threaded portion of the actuation rod of the rotary manipulation member is configured to move within the thread of the internally threaded actuation part when the user applies a clockwise or anti-clockwise screwing movement to the head of the rotary manipulation member.
the internally threaded actuation part comprises a part body comprising a bore, i.e. a continuous or blind orifice or the like, the inner wall of which is tapped, i.e. carries a thread . . .
the threaded actuation part comprises an internal thread complementary to the thread of the threaded portion of the actuation rod of the rotary manipulation member.
the internally threaded actuation part is configured to be moved in translation when the actuation rod of the rotary manipulation member acts on the internally threaded actuation part.
the internally threaded actuation part is configured to produce an angular movement, that is to say a pivoting, of the clamp element relative to the clamp body, and a movement of the second jaw towards or away from the first jaw, depending on the direction of actuation of the actuation rod by the user, when the actuation rod of the rotary manipulation member acts on the internally threaded actuation part.
the internally threaded actuation part is made of metal, for example stainless steel.
the internally threaded actuation part constitutes an intermediate part forming the link between the actuation rod of the manipulation member and the pivoting clamp element. the internally threaded actuation part constitutes an intermediate part (i.e. linking part) by which the rotary movement applied by the user to the manipulation member and to the actuation rod can be converted into a translational movement, so that said actuation part can cooperate with the pivoting clamp element in order to make it pivot about the axis XX.
the pivoting takes place about a pivot axis (XX).
the first clamping zone of the first jaw has a semi-circular profile, that is to say it has a semi-cylindrical or similar shape, and the second clamping zone of the second jaw has a linear profile, that is to say it is flat or plane, or vice versa.
the first jaw and the second jaw are shaped to allow a vice-like grip of a support of polygonal or circular cross section, in particular a rod of square or rectangular cross section, or a bar of circular cross section, and to ensure a firm, i.e. solid, hold thereof.
the head of the rotary manipulation member is hollow, that is to say it comprises an internal chamber.
the head of the manipulation member is made of polymer or metal, for example of aluminium or an aluminium alloy.
the torque-limiting system is arranged in a chamber or an internal volume of the head of the manipulation member.
the torque-limiting system comprises a stack of several spring washers comprising fewer than 12 spring washers, preferably from 2 to 8 spring washers.
a holding means, such as a screw or the like, makes it possible to secure the stack of spring washers to the actuation rod.
the stack of spring washers is held between a head of the holding means, such as a screw head or the like, and a pressure washer, also called a lock washer.
the stack of spring washers presses against the pressure washer arranged in the head of the rotary manipulation member, preferably exerting there an axial thrust force (FdP) (axis AA).
the spring washers are elastic.
the head of the rotary manipulation member comprises an internal annular shoulder arranged, i.e. sandwiched, between the pressure washer and a base element integral with the actuation rod.
the annular shoulder is traversed by at least one pin housing comprising a movable part movable in translation within said pin housing.
the movable part is a pin or the like.
the annular shoulder is traversed by several pin housings, each comprising a movable part, for example 2 or 3 pin housings, or more.
the pin housings are arranged and/or angularly distributed on the annular shoulder.
the pin housing(s) is (are) a drilled hole, preferably of axis AA, passing through the annular shoulder.
the annular shoulder has a ring shape.
the base element comprises at least one blind housing provided on an upper annular surface of the base element situated facing the shoulder.
the base element comprises several blind housings arranged on the upper annular surface of the base element.
the torque-limiting system can adopt at least one engaged position and one disengaged position, depending on the maximum clamping torque of the torque-limiting system, i.e. depending on whether or not the maximum clamping torque has been reached.
the movable part is partially inserted into the at least one blind housing of the base element when the torque-limiting system is in said at least one engaged position.
in the engaged position, the head of the rotary manipulation member is coupled to the base element, via the annular shoulder, so that a rotational movement, i.e. clockwise or anti-clockwise, applied by the user to the head of the rotary manipulation member simultaneously drives a rotation of the head of the rotary manipulation member and of the base element, since they are integral/coupled to each other.
the movable part is positioned outside the at least one blind housing of the base element when the torque-limiting system is in the disengaged position.
in the disengaged position, the head of the rotary manipulation member is decoupled from the base element, so that a rotational movement (i.e. clockwise or anti-clockwise) applied by the user to the head of the rotary manipulation member does not drive/no longer drives the base element, that is to say the head of the rotary manipulation member rotates freely without cooperating with the base element.
in the disengaged position, said at least one movable part pushes the pressure washer towards the stack of spring washers so as to move the pressure washer away from the annular shoulder of the head of the rotary manipulation member.
the apparatus is an apparatus for supplying NO gas, in particular an NO/N2 gas mixture, intended to be connected to a patient circuit of a medical ventilator supplying a respiratory gas, in particular an N2/O2 gas mixture or air.
the apparatus further comprises, arranged in the shell, an internal gas circuit for conveying the gas, means for controlling the flow rate of the gas conveyed by the internal gas circuit, and control means for controlling the flow rate control means.
the internal gas circuit comprises one or more gas passages.
the flow rate control means comprise one or more controlled valves, in particular one or more solenoid valves.
the operating means comprise at least one microprocessor.
the microprocessor(s) is (are) arranged on at least one electronic card.
the microprocessor(s) implement(s) at least one algorithm.
it comprises first means of connection to one or more gas sources, such as gas cylinders, i.e. containing NO/N2 mixture and/or oxygen.
it comprises second means of connection to the patient circuit of the medical ventilator.
it comprises third means of connection to a flow sensor arranged on or in the patient circuit of the medical ventilator.
it comprises a display screen, preferably carried by the front face of the shell of the apparatus.
it comprises electrical power supply means, such as one or more mains connection cables (110/220V) and/or an electric socket.
alternatively, the apparatus is a medical ventilator supplying air or an air/oxygen mixture.

### BRIEF DESCRIPTION OF THE VIEWS OF THE DRAWINGS

The invention will now be better understood from the following detailed description given by way of illustration but without limitation, with reference to the appended figures, in which:

FIG. 1 shows schematically an embodiment of a fixing device for a medical apparatus according to the present invention, in particular for an NO supply apparatus,

FIG. 2 shows schematically the shape of the jaws (side view) of the fixing device of FIG. 1,

FIG. 3 shows schematically (front view) an embodiment of a medical apparatus for supplying NO, equipped with the clamping device according to the present invention,

FIG. 4 is a side view of the apparatus of FIG. 3, showing the fixing device of FIG. 1,

FIG. 5 shows schematically (cross-sectional view) an embodiment of the rotary member incorporating a torque limiter of the clamping device according to the present invention of FIG. 1,

FIG. 6A is an enlarged view of a part of the rotary member incorporating the torque limiter of the clamping device of FIG. 5,

FIG. 6b is a sectional view along the plane B-B of FIG. 6A, and

FIG. 7 to FIG. 9 show schematically the operation of the torque limiter of the clamping device of FIG. 5 and FIG. 6A.

### DETAILED DESCRIPTION

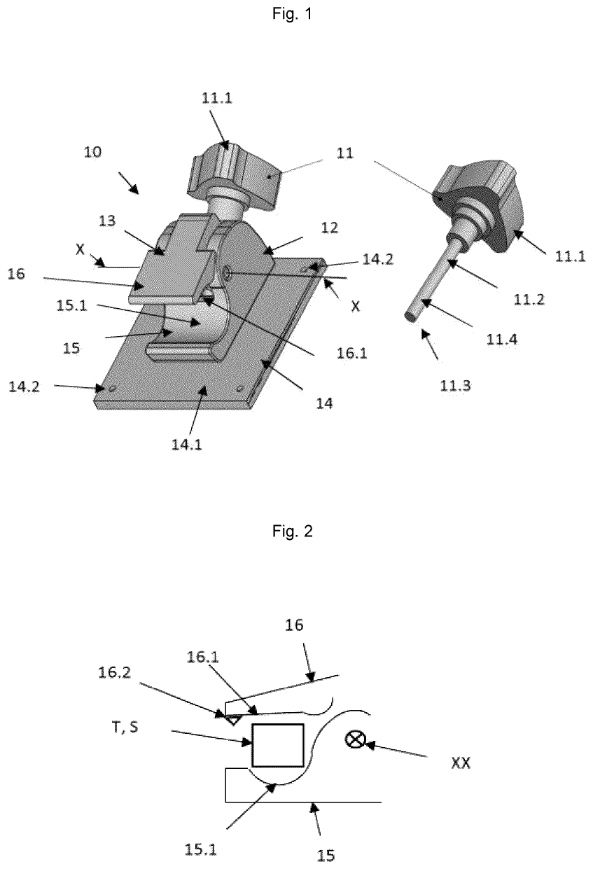

FIG. 1 shows schematically an embodiment of a fixing device 10 for a medical apparatus 1 according to the present invention, in particular for an NO supply apparatus.

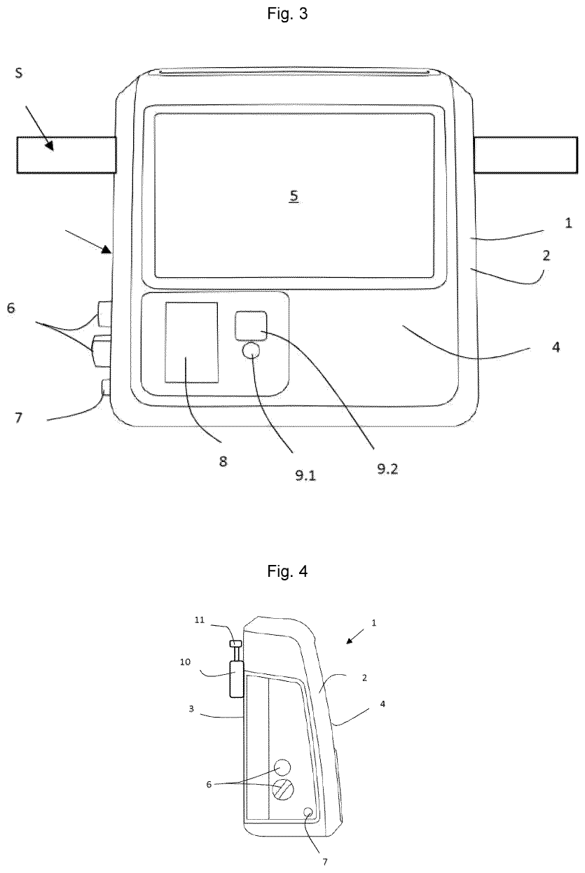

The fixing device 10 according to the invention of FIG. 1 is intended to equip a medical apparatus 1, as shown schematically in FIG. 3 and FIG. 4, in particular an apparatus for supplying NO, i.e. an NO/nitrogen mixture, for example of the type described by EP-A-3906955 or the one marketed by Air Liquide Healthcare® under the trade name SoKinox®.

It is designed to allow the medical apparatus 1 to be attached and fixed to an elongate support S, such as a T-rod of rectangular or square external section (i.e. perimeter), or a bar of cylindrical shape, that is to say of circular external section (i.e. perimeter), in particular a horizontal rod or a horizontal or vertical bar, as illustrated in FIG. 3.

FIG. 3 and FIG. 4 show schematic views of the front face 4 and rear face 3 of an embodiment of a medical apparatus 1 for supplying NO, equipped with the fixing device 10 according to the present invention, which medical apparatus 1 is shown attached to an elongate support S in FIG. 3.

The fixing device 10 is carried by the rear face 3 of the external casing 2, that is to say of the rigid peripheral shell, of the medical apparatus 1 as shown schematically in FIG. 4, for example a rigid polymer casing 2.

The front face 4 of the medical apparatus 1 for supplying NO comprises an information display screen 5, advantageously a touch-sensitive panel screen, preferably with colour display, for displaying information, data, graphs, measurements or any other data useful to the user, typically a healthcare provider, such as a doctor. The information display screen 5 with touch panel also allows the user to make choices, adjustments, selections, validations or the like via one or more touch keys and/or selection menus or the like displayed on the screen 5.

The medical apparatus 1 for supplying NO also comprises one or more adjustment knobs 6 or the like, for example arranged on one of the side faces of the apparatus 1, and also one or more connectors or fittings 7 for connecting flexible pipes that carry gas, such as an NO/N2 mixture, oxygen or mixtures thereof.

Preferably, it also comprises an inlet of a gas sampling line 8 with a device of the water trap type, a flow measurement inlet 9.2 and an injection outlet 9.1 for supplying the NO/N2 mixture.

The fixing device 10, arranged on the rear face 3 of the casing 2 of the medical apparatus 1, comprises a rotary manipulation member 11, a fixed clamp body 12 and a movable clamp element 13, which are carried by a support plate 14. Their operation is set out in detail below.

In the embodiment of FIG. 1, the support plate 14 comprises one or more plates 14.1 forming a base, for example of metal, which is pierced with several holes 14.2 for the passage of screws or the like serving for the screw-fastening of the support plate 14 to the rear face 3 of the casing 2 of the medical apparatus 1, in particular an apparatus for supplying NO.

In this case, a part of the clamp body 12 is configured to form a first clamp jaw 15, and a part of the movable clamp element 13 is configured to form a second clamp jaw 16.

The first and second jaws 15, 16 are situated facing each other, that is to say arranged face to face, in order to form a clamp that makes it possible to receive elongate supports S of different shapes, namely, without distinction, a support of polygonal cross section, e.g. square or rectangular, or of circular cross section, typically a T-rod carried by a wall of a vehicle or building, or a cylindrical bar of a stretcher, hospital bed or the like. By virtue of a clamping action in which the first and second jaws 15, 16 are brought together, the elongate support S, whether of polygonal or circular cross section, can be effectively held between said jaws 15, 16, by being sandwiched or held in a vice between said jaws 15, 16.

In order to improve the grip of the elongate support S and its firm hold between them, the first and second jaws 15, 16 are shaped so as to have clamping zones 15.1, 16.1 of particular shapes.

Thus, in the embodiment proposed in FIG. 1, the first clamping zone 15.1 of the first jaw 15 has a semicircular profile or section, that is to say it has a semi-cylindrical or similar shape, whereas the second clamping zone 16.1 of the second jaw 16 has a rectilinear profile, i.e. straight (not curved), that is to say it has a flat or plane surface. These profiles are more visible in FIG. 2.

According to one embodiment, the second clamping zone 16.1, namely the plane surface visible in FIG. 2, is preferably terminated by a rim 16.2 making it possible to avoid or limit any untimely exit by sliding of the elongate support S, such as a polygonal T-rod or a cylindrical bar.

Although less preferable, according to another embodiment the profiles of the first and second clamping zones 15.1, 16.1 could be reversed.

In all cases, these particular profiles of these first and second clamping zones 15.1, 16.1 make it possible to perform effective clamping of the elongate support S, whatever its shape, that is to say not only a T-rod of polygonal cross section, e.g. square or rectangular, as shown schematically in FIG. 2, but also a cylindrical bar, i.e. of circular, oval or ellipsoid cross section. Indeed, the semi-circular profile of the first clamping zone 15.1 of the first jaw 15 is well adapted to supports S of the cylindrical bar type, whereas the linear profile (i.e. plane) of the second clamping zone 16.1 of the second jaw 16 is well adapted to supports S such as a T-rod of polygonal section or the like. Therefore, their combination generates a ‘synergistic’ effect, making it possible to firmly maintain the two types of supports S.

Furthermore, according to the present invention, the rotary manipulation member 11, which cooperates with the movable clamp element 13, comprises a head 11.1 and an actuation rod 11.2, which is threaded 11.4 at its free end 11.3.

The head 11.1 of the rotary manipulation member 11 is intended to be grasped manually by the user and actuated in the clockwise direction, for example, in order to effect clamping by bringing the first and second clamping zones 15.1, 16.1 closer to each other, as a result of a pivoting of the second jaw 16 in the direction of the fixed first jaw 15, or, conversely, unclamping in the anti-clockwise direction.

In other words, the actuation of the head 11.1 of the rotary manipulation member 11 makes it possible to move the second jaw 16 towards or away from the first jaw 15.

To do this, the actuation rod 11.2 comprises a threaded portion 11.4 (i.e. with a peripheral thread), preferably located at a free end 11.3, which will cooperate, when manipulated by the user, with an internally threaded actuation part (not visible) which is integral with the movable, i.e. pivoting, clamp element 13 and which acts on the latter to cause it to pivot.

During screwing or unscrewing, the threaded portion 11.4 of the actuation rod 11.2 cooperates with the internal thread of the internally threaded actuation part, since their threads are configured to be complementary to each other, i.e. same threads.

The internal thread of the actuation part is a bore, i.e. orifice or the like, formed through the actuation part, in the peripheral wall of which an internal thread is formed. The internally threaded actuation part is therefore a machined intermediate part, for example drilled and threaded, so as to have a internal thread in its bore.

The movement, preferably in translation, of the internally threaded actuation part will generate an angular movement, that is to say a pivoting (about axis XX), of the clamp element 13 with respect to the clamp body 12, thus a movement of the second jaw 16 towards or away from the fixed first jaw 15, depending on the direction in which the actuation rod 11.2 is actuated, via its head 11.1, by the user, that is to say screwing or unscrewing of the head 11.1 of the rotary manipulation member 11.

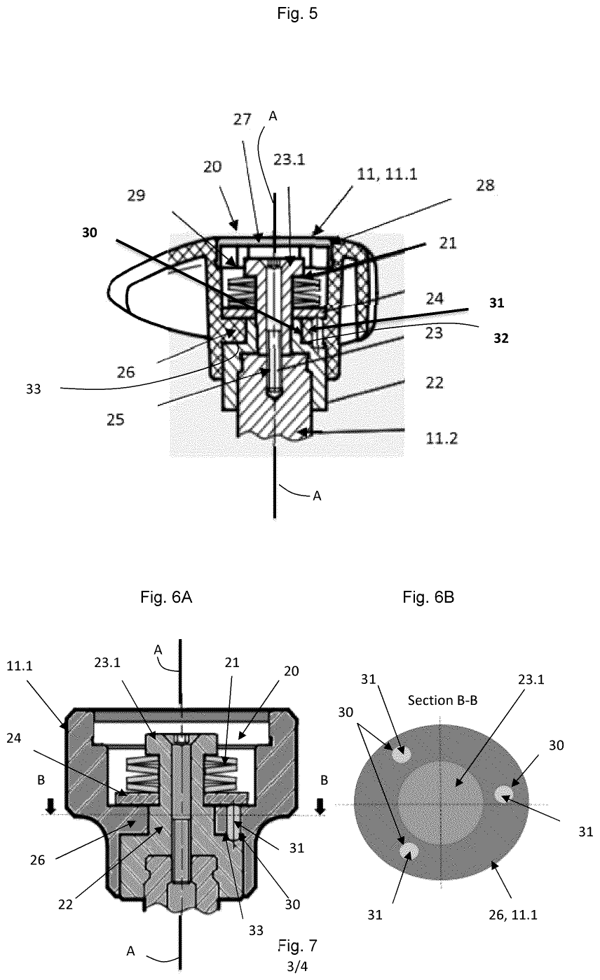

Advantageously, as shown schematically in FIG. 5, FIG. 6A and FIG. 6b, the rotary member 11, in particular the head 11.1 which is (at least partly) hollow, incorporates a torque-limiting system 20 which operates by friction via a stack of spring washers 21, such as ‘Belleville’ washers or the like.

Typically, a stack of 2 to 8 spring washers 21. These spring washers 21 are positioned on a base element 22 that covers, i.e. is arranged on, the upper end of the actuation rod 11.2. A pressure washer 24, also called a lock washer, is sandwiched between the stack of spring washers 21 and the base element 22. The base element 22 is coupled to the actuation rod 11.2, and the assembly consisting of base element 22 and actuation rod 11.2 can be driven in rotation about the axis AA, as is explained below and illustrated in FIG. 5 and FIG. 6A, in particular during screwing or unscrewing of the head 11.1 of the rotary manipulation member 11 by the user.

The head 11.1 of the rotary manipulation member 11 comprises an internal annular shoulder 26, namely an internal wall, preferably of annular shape, projecting radially (i.e. directed towards the axis AA) into the head 11.1, as is illustrated in FIG. 5 and FIG. 6A. This internal shoulder 26 is sandwiched between the pressure washer 24 and the base element 22.

Preferably, this internal shoulder 26 has a ring shape. The internal shoulder 26 is traversed, preferably axially (axis AA), by at least one pin housing 30, preferably several pin housings 30 distributed over the shoulder 26, in which pin housing(s) a movable part 31, in particular a pin or the like, is housed, that is to say a movable part 31 is arranged in each pin housing 30, as is illustrated in FIG. 5 and FIG. 6A.

FIG. 6b, which is a sectional view along B-B of FIG. 6A, shows an embodiment in which there are several pin housings 30, which each comprise a movable part 31 and which are arranged and angularly distributed on the annular shoulder 26, that is to say in the shape of a ring.

The pin housing or housings 30 are bores passing axially through the ring-shaped internal shoulder 26. The movable part(s) 31 is (are) configured to move axially, i.e. in translation, within said pin housing 30.

The base 22 further comprises at least one blind housing 32, preferably several blind housings 32, formed on an upper annular surface 33 situated facing the shoulder 26, such that the movable part(s) 31 can be housed in at least one blind housing 32, when the torque-limiting system 20 is in an engaged position and capable of driving the base element 22 in rotation at the same time as the head 11.1 of the rotary manipulation member 11 comprising the shoulder 26. In other words, when one or more movable parts 31 are partially inserted in one or more blind housings 32, the head 11.1 of the rotary manipulation member 11 and the base element 22 are secured to each other and are movable “as a unit” in rotation about the axis AA, for example when the user exerts a clockwise clamping movement of the rotary manipulation member 11 so as to bring the first and second jaws 15, 16 closer to each other.

The pressure washer 24 presses against the movable part 31 so as to push it towards the base element 22, as long as the torque-limiting system 20 does not “trigger”, as is explained below, in the event of clamping beyond the maximum torque.

The assembly comprising the spring washers 21, the pressure washer 24 and the base 22 cooperates with the actuation rod 11.2 and is fixed thereto by a holding means 23, such as a screw or the like, which is secured to the upper end of the actuation rod 11.2, for example fixed by screwing in a housing 25 provided in the upper end of the actuation rod 11.2.

As can be seen in FIG. 5 and FIG. 6A, the spring washers 21 are held between the head 23.1 of the holding means 23, such as a screw, and the pressure washer 24. More precisely, the holding means 23, such as a screw or the like, passes through the spring washers 21, the pressure washer 24 and the base 22.

In order to enable the torque-limiting system 20 to be mounted in the head 11.1 which is hollow, i.e. comprises an internal chamber or volume 29, a removable cover 27 is provided which closes an opening 28 provided on the head 11.1, which opening 28 gives access to the internal volume 29 of the head 11.1 of the rotary member 11.

In order to perform the clamping on a bar, the head 11.1 of the rotary member 11 has to be turned clockwise, for example.

As is shown schematically in FIG. 5, FIG. 6A and FIG. 7, during clamping in the clamping direction (SdS), the head 11.1 of the rotary member 11 drives the base 22, and therefore also the actuation rod 11.2, in rotation in the clamping direction (SdS), typically the clockwise direction. The spring washers 21, i.e. calibrated elastic washers, exert a thrust force (FdP), that is to say exert a force directed towards the base element 22, and then push the pressure washer 24 back towards the base element, which simultaneously pushes back the movable part(s) 31, such as a pin or the like, in the blind housing 32 carried by the upper annular surface 33 of the base element 22.

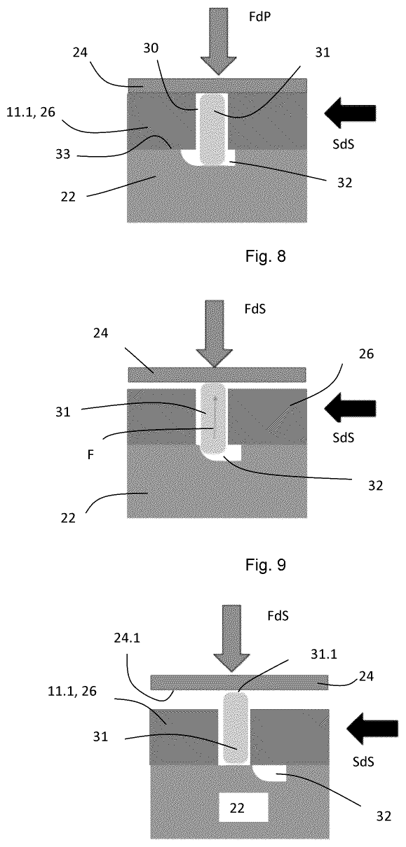

As long as the clamping torque is below a maximum value, the movable part 31 is or remains partially inserted in a blind housing 32, the head 11.1 of the rotary manipulation member 11 and the base element 22 are secured to each other and are movable “as a unit” in rotation about the axis AA, that is to say they are in an engaged position, as is illustrated in FIG. 7 and FIG. 8. In fact, the nominal clamping torque is defined by the frictional forces generated by the elastic deformation of the spring washers 21 as they are clamped, that is to say as they are progressively crushed between the head 23.1 of the holding means 23, i.e. screws or the like, and pressure washer 24.

It will be seen that as the clamping continues, as is illustrated in FIG. 8, the pressure forces (FdP) applied by the spring washers 21 tend to decrease due to the progressive deformation of the spring washers 21, which generates an axial movement of the movable part 31 in the housing 30 (see arrow F directed upward in FIG. 8), moving away from the base element 22, thus causing its progressive exit from the blind housing(s) 32 in which it is partially inserted. In fact, the pressure forces applied by the spring washers 21 then become insufficient to continue pushing back the pressure washer 24, and the latter then tends to move away from the upper surface of the shoulder 26.

When the maximum clamping torque is reached, as is shown in FIG. 9, the torque-limiting system 20 of the head 11.1 triggers an “over-engagement”, which is to say that, beyond a certain stress, the pressure forces (FdP) of the elastic spring washers 21, therefore on the part 24, are no longer sufficient to maintain the engagement function, i.e. the engaged position, which drives the entire system in rotation. The pressure washer 24 serving as a drive part then turns idly because the movable part(s) 31, that is to say the pin(s) or the like, is (are) no longer inserted in the blind housing(s) 32 carried by the upper annular surface 33 of the base element 22, which then decouples the head 11.1 of the rotary member 11 from the base 22, and then allows the head 11.1 of the rotary member 11 to rotate freely about the axis AA without driving the base element 22.

In other words, during clamping, the user cannot exceed the maximum torque because, once the maximum torque has been reached, the head 11.1 of the rotary member 11 will turn on idle. It then becomes impossible to effect clamping beyond the maximum torque. The pressure washer 24 and the shoulder 26 of the head 11.1 of the rotary member 11 are then disengaged from each other, that is to say decoupled.

Such an arrangement thus makes it possible to ensure optimum screwing.

Preferably, a sound audible to the user, such as a “click”, and created by the disengagement of the system confirms to the user that the maximum torque has been reached. To do this, notches or the like can be provided on the lower face 24.1 of the pressure washer 24 and will then rub against the head 31.1 of the (or each) movable part 31, making the audible sound.

Conversely, unclamping can be effected by turning the head 11.1 of the rotary member 11 in the opposite direction, for example the anti-clockwise direction. The spring washers 21 will then be progressively less compressed and will push the pressure washer 24 progressively towards the base element 22. The movable part(s) 31, such as a pin or the like, will then be able to be inserted again into the blind housing(s) 32 carried by the upper annular surface 33 of the base element 22, so as to again couple the base 22 and the head 11.1 of the rotary member 11, via the shoulder 26, and thus allow them to be driven in simultaneous rotation in order to unscrew the rotary clamping member 11 and thus move the first and second jaws 15, 16 away from each other, that is to say perform unclamping.

In general, according to the invention, the torque-limiting system 20 can therefore adopt at least two distinct positions comprising an engaged position and a disengaged position, depending on whether the maximum clamping torque is reached or not.

By virtue of this torque-limiting system 20, it is possible to guarantee effective and reproducible, i.e. identical, clamping, irrespective of which user is operating the clamping, since the clamping is only carried out until the “disengage” function is triggered, which occurs automatically as soon as a maximum or ‘optimum’ clamping threshold is reached, i.e. neither too tight nor loose or insufficiently tightened. The clamping threshold is preferably pre-set, for example at the factory, and cannot be modified by the user, so as to limit the risk of incorrect setting by the user, which may lead to excessively strong or insufficient clamping.

In general, the fixing device 10 according to the invention is particularly well suited for use on any gas supply apparatus, whether it be gaseous NO, in particular an NO/N2 gas mixture, or another gas, for example a medical ventilator supplying air, oxygen or an air/O2 mixture, or even another medical gas,

When the apparatus is an NO supply apparatus, it may be intended to be connected to the patient circuit of a medical ventilator supplying a respiratory gas, in particular an N2/O2 mixture or air, so as to obtain one or more therapeutic gas mixtures based on NO, O2 and N2, or even other compounds. The fixing device 10 according to the invention makes it possible to hold the apparatus for supply of gas, such as NO or another gas, firmly attached to an elongate support S, such as a bed bar, stretcher bar or the like, or a T-rod, in particular in a transport vehicle or the like, before, during and after treatment of a patient by inhalation of therapeutic gas, including during movement of the patient from one site to another, for example within a hospital building or in a transport vehicle, such as an ambulance, a plane, a helicopter or the like.

What is claimed is:

1. Medical apparatus (1) for supplying gas, comprising a shell (2) having an outer wall (3) and a fixing device (10), arranged on the outer wall (3) of said shell (2), for fixing the latter to an elongate support (S), wherein the fixing device (10) comprises:
a clamp body (12) and a clamp element (13) movable by pivoting (XX) relative to each other,
at least part of the clamp body (12) being configured as a first jaw (15) and at least part of the movable clamp element (13) being configured as a second jaw (16), said first and second jaws (15, 16) being arranged face to face in order to form a clamp, and
a rotary manipulation member (11), which can be actuated in rotation by a user, cooperating with the clamp element (13), when actuated, in order to move the clamp element (13) with respect to the clamp body (12) and move the first jaw (15) towards or away from the second jaw (16),
and wherein:
the rotary manipulation member (11) comprises a head (11.1) intended to be grasped manually by the user, and an actuation rod (11.2) comprising a threaded portion (11.4), and
an internally threaded actuation part cooperates with the threaded portion (11.4) of the actuation rod (11.2) and with the clamp element (13) in order to effect the pivoting (XX) of the clamp element (13) relative to the clamp body (12) in such a way as to move said first and second jaws (15, 16) towards or away from each other,
characterized in that the rotary member (11) further comprises a torque-limiting system (20) arranged in the head (11.1) of the rotary manipulation member (11), said torque-limiting system (20) comprising a stack of spring washers (21) cooperating with a pressure washer (24).

2. Apparatus according to claim 1, characterized in that the clamp body (12) and the clamp element (13) are carried by a support plate (14), said support plate (14) being fixed to the outer wall (3) of said shell (2).

3. Apparatus according to claim 1, characterized in that the threaded portion (11.4) of the actuation rod (11.2) of the rotary manipulation member (11) is configured to move within the thread of the internally threaded actuation part when the user applies a clockwise or anti-clockwise screwing movement to the head (11.1) of the rotary manipulation member (11).

4. Apparatus according to claim 1, characterized in that the internally threaded actuation part is configured to be moved in translation when the actuation rod (11.2) of the rotary manipulation member (11) acts on the internally threaded actuation part.

5. Apparatus according to claim 1, characterized in that the internally threaded actuation part is configured to produce an angular movement (XX) of the clamp element (13) relative to the clamp body (12), and a movement of the second jaw (16) towards or away from the first jaw (15), depending on the direction of actuation of the actuation rod (11.2) by the user, when the actuation rod (11.2) of the rotary manipulation member (11) acts on the internally threaded actuation part.

6. Apparatus according to claim 1, characterized in that the first clamping zone (15.1) of the first jaw (15) has a semi-circular profile, and the second clamping zone (16.1) of the second jaw (16) has a linear profile, or vice versa.

7. Apparatus according to claim 1, characterized in that the torque-limiting system (20) comprises a stack of fewer than 12 spring washers, preferably from 2 to 8 spring washers.

8. Apparatus according to claim 1, characterized in that the stack of spring washers (21) presses against the pressure washer (24) arranged in the head (11.1) of the rotary manipulation member (11).

9. Apparatus according to claim 1, characterized in that the head (11.1) of the rotary manipulation member (11) comprises an internal annular shoulder (26) arranged between the pressure washer (24) and a base element (22) integral with the actuation rod (11.2).

10. Apparatus according to claim 9, characterized in that the annular shoulder (26) is traversed by at least one pin housing (30) comprising a movable part (31) movable in translation within said pin housing (30), preferably several pin housings (30) each comprising a movable part (31).

11. Apparatus according to claim 9, characterized in that the base element (22) comprises at least one blind housing (32) formed on an upper annular surface (33) of the base element (22) situated facing the shoulder (26).

12. Apparatus according to claim 10, characterized in that the movable part (31) is partially inserted in said at least one blind housing (32) of the base element (22) when the torque-limiting system (20) is in at least one engaged position, in which the head (11.1) of the rotary manipulation member (11) is coupled to the base element (22) via the annular shoulder (26), such that a rotational movement applied by the user to the head (11.1) of the rotary manipulation member (11) simultaneously drives a rotation of the head (11.1) of the rotary manipulation member (11) and of the base element (22).

13. Apparatus according to claim 10, characterized in that the movable part (31) is positioned outside said at least one blind housing (32) of the base element (22) when the torque-limiting system (20) is in at least one disengaged position, in which the head (11.1) of the rotary manipulation member (11) is decoupled from the base element (22), such that a rotational movement applied by the user to the head (11.1) of the rotary manipulation member (11) does not drive the base element (22).

14. Apparatus according to claim 13, characterized in that, in the disengaged position, said at least one movable part (31) pushes the pressure washer (24) towards the stack of spring washers (21) so as to move the pressure washer (24) away from the annular shoulder (26) of the head (11.1) of the rotary manipulation member (11).

15. Apparatus according to claim 13, characterised in that a holding means (23) makes it possible to secure the stack of spring washers (21) to the actuation rod (11.2), the stack of spring washers (21 preferably being held between a head (23.1) of the holding means (23) and the pressure washer (24) arranged in the head (11.1) of the rotary manipulation member (11).
